SEQUENCE LISTING (20 sequences)

SEQ ID NO: 1 - DNA, 567 bp, Homo sapiens
atgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 2 - DNA, 567 bp, Homo sapiens
atgactgaatataaacttgtggtagttggagctgatggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 3 - DNA, 567 bp, Homo sapiens
atgactgaatataaacttgtggtagttggagcttgtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 4 - DNA, 567 bp, Homo sapiens
atgactgaatataaacttgtggtagttggagctgttggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 5 - DNA, 567 bp, Homo sapiens
atgactgaatataaacttgtggtagttggagctcgtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 6 - DNA, 567 bp, Homo sapiens
atgactgaatataaacttgtggtagttggagctagtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 7 - DNA, 567 bp, Homo sapiens
atgactgaatataaacttgtggtagttggagctgctggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaa

SEQ ID NO: 8 - DNA, 21 bp, Artificial Sequence
  Synthetic oligonucleotide
  DNA (20)..(21)
guuggagcugguggcguagtt

SEQ ID NO: 9 - DNA, 21 bp, Artificial Sequence
  Synthetic oligonucleotide
  DNA (20)..(21)
cuacgccaccagcuccaactt

SEQ ID NO: 10 - DNA, 21 bp, Artificial Sequence
  Synthetic oligonucleotide
  DNA (20)..(21)
guuggagcugauggcguagtt

SEQ ID NO: 11 - DNA, 21 bp, Artificial Sequence
  Synthetic oligonucleotide
  DNA (20)..(21)
cuacgccaucagcuccaactt

SEQ ID NO: 12 - DNA, 21 bp, Artificial Sequence
  Synthetic oligonucleotide
  DNA (20)..(21)
guuggagcuuguggcguagtt

SEQ ID NO: 13 - DNA, 21 bp, Artificial Sequence
  Synthetic oligonucleotide
  DNA (20)..(21)
cuacgccacaagcuccaactt

SEQ ID NO: 14 - protein, 11 aa, Human immunodeficiency virus
YGRKKRRQRRR

SEQ ID NO: 15 - protein, 17 aa, Artificial Sequence
  Synthetic peptide
GALFLGFLGAAGSTMGA

SEQ ID NO: 16 - protein, 12 aa, Artificial Sequence
  Synthetic peptide
KETWWETWWTEW

SEQ ID NO: 17 - DNA, 21 bp, Artificial Sequence
  Synthetic oligonucleotide
gaggcctgctgaaaatgactg

SEQ ID NO: 18 - DNA, 21 bp, Artificial Sequence
  Synthetic oligonucleotide
ttactacttgcttcctgtagg

SEQ ID NO: 19 - DNA, 20 bp, Artificial Sequence
  Synthetic oligonucleotide
aaatctggcaccacaccttc

SEQ ID NO: 20 - DNA, 20 bp, Artificial Sequence
  Synthetic oligonucleotide
ggggtgttgaaggtctcaaa

(19) United States
(12) Patent Application Publication
(10) Pub. No.: US 2022/0251565 A1
(43) Pub. Date: Aug. 11, 2022

(54) Compositions and Methods for Reducing Perineural Invasion and Pain

(71) Applicant: Silenseed Ltd., Modiin (IL)

(72) Inventors: Amotz Shemi, Modiin (IL); Shay ROTKOPF, Modiin (IL); Rachel MALKA GABAI, Modiin (IL)

(73) Assignee: Silenseed Ltd., Modiin (IL)

(21) Appl. No.: 17/611,927

(22) PCT Filed: May 17, 2020
(86) PCT No.: PCT/IL2020/050537

§ 371 Date: Nov. 17, 2021

Related U.S. Application Data
(60) Provisional application No. 62/849,166, filed on May 17, 2019.

(51) Int. Cl. C12N 15/113; A61P 35/04

(52) U.S. Cl. CPC C12N 15/1135; C12N 2310/14; A61P 35/04

(57) ABSTRACT

Provided herein are compositions for reducing regional perineural invasion and pain in a subject. Methods of reducing regional perineural invasion, and associated pain are also described.

### CROSS REFERENCE TO RELATED APPLICATIONS

Benefit is claimed to U.S. Provisional Patent Application No. 62/849,166, filed May 17, 2019, the contents of which are incorporated by reference herein in their entirety.

### FIELD

This disclosure relates to compositions for reducing perineural invasion and pain in a subject. Methods of reducing perineural invasion, and associated pain, are also described.

### BACKGROUND

Metastasis of solid tumors is a hallmark of cancer progression, and the spread of a cancer from its tumor of origin to relatively distant locations in a patient is often a sign of poor patient prognosis. Perineural invasion (PNI) is a distinct form of tumor metastasis involving the regional migration of tumor-originating cells in relation to, along, and within the epineural, perineural, and endoneural layers of nerve fibers (Gasparini et al. Cancers 11:893, 2019). Although often detected in early stages of a cancer, such as pancreatic cancer, and before the appearance of more distant metastases, PNI is often associated with tumor recurrence, pain, and overall poor prognosis for survival.

KRAS is a GTPase protein encoded by the Kirsten rat sarcoma 2 viral oncogene homolog oncogene, which belongs to the family of RAS proteins. Mutations in KRAS, have long been associated with multiple cancer types, and particularly pancreatic cancer. The KRAS mutant allele G12D has previously been targeted for treatment of solid tumors and to inhibit cellular migration (see U.S. Pat. No. 9,080,173 and US Patent Publication No. 2014/0314854). The RNA interference (RNAi) compositions described therein demonstrated reduction in tumor size and inhibition of tumor growth and cellular migration. However, the specific inhibition of PNI, a distinct, non-inherent form of cancer metastasis, by mutant KRAS RNAi agents was not shown or previously suggested.

### SUMMARY

Provided herein are compositions that include an antisense oligonucleotide agent that targets at least one KRAS mutant allele selected from KRAS G12D, KRAS G12C, KRAS G12V, KRAS G12R, KRAS G12S, and KRAS G12A, for use in inhibiting or preventing regional perineural invasion or pain associated with such perineural invasion by a solid tumor in a subject. Use of such compositions in the preparation of a medicament for inhibiting or preventing such regional perineural invasion or pain is also described.

Also described herein are methods for reducing regional perineural invasion by a solid tumor by administering to a subject in need thereof (i.e. a subject diagnosed with a solid tumor or suspected of having a solid tumor or predisposed to having a solid tumor) a therapeutically effective amount of a composition that includes an antisense oligonucleotide agent that targets at least one KRAS mutant allele selected from the group consisting of KRAS G12D, KRAS G12C, KRAS G12V, KRAS G12R, KRAS G12S, and KRAS G12A. Similar methods for reducing pain resultant from perineural invasion by a solid tumor are also described.

The foregoing and other objects, features, and advantages will become more apparent from the following detailed description, which proceeds with reference to the accompanying figures.

### BRIEF DESCRIPTION OF THE DRAWINGS

FIGS. 1A-1D show decrease in KRAS RNA transcript levels after siRNA transfection. MIA PaCa-2 cells were transfected with either siG12D (G12D), siG12C (G12C), anti-Luciferase siRNA (LUC), scrambled siRNA (SCR) (the last two as negative controls), or mock transfected. KRAS mRNA levels were measured using Real-Time PCR. The results are presented as relative mRNA level (average of three samples) ±standard deviation. P-values were calculated using Student's t-test. FIGS. 1A and 1B show effect of siG12D. FIGS. 1C and 1D show effect of siG12C.

FIGS. 2A-2D show decrease in KRAS protein levels after siRNA transfection. MIA PaCa-2 cells were transfected with either siG12C (G12C) or scrambled siRNA (SCR) as a negative control. FIGS. 2A and 2C: KRAS protein levels were measured after 48 and 72 hours using Western Blot. Anti-Beta Actin antibody was used as a loading control. FIGS. 2B and 2D: Quantification of the results shown in A and C. The results are presented as band pixel area (average of three samples) ±standard deviation. P-values were calculated using Student's t-test.

FIGS. 3A-3C show the MIA PaCa-2—DRG co-culture assay. MIA PaCa-2 colonies were plated in proximity to a freshly-isolated mouse DRG, 48 hours after transfection with either siG12D, siG12C, or scrambled siRNA as a negative control. The co-cultures were monitored for migration of the MIA PaCa-2 cells towards the DRG. FIG. 3A: Low magnification image showing the DRG surrounded by four MIA PaCa-2 colonies. FIG. 3B: Representative images of the different treatments as noted, at day 13 of co-culturing. FIG. 3C: Quantitative summary of co-culture assay, comparing PNI of MIA PaCa-2 cells after transfection with either siG12D, siG12C, or scrambled siRNA.

FIG. 4 shows a representative image of the described MIA PaCa-2—DRG co-culture assay at 20 days incubation following transfection with negative control scrambled siRNA. Bottom panel is a magnified section indicated by the box in the top panel.

FIG. 5 shows a representative image of the described MIA PaCa-2—DRG co-culture assay at 20 days incubation following transfection with siRNA targeting KRAS G12C. Bottom panel is a magnified section indicated by the box in the top panel.

FIG. 6 shows a representative image of the described MIA PaCa-2—DRG co-culture assay at 20 days incubation following transfection with siRNA targeting KRAS G12D. Bottom panel is a magnified section indicated by the box in the top panel.

### BRIEF DESCRIPTION OF THE DESCRIBED SEQUENCES

The nucleic and/or amino acid sequences provided herewith are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file named 2142 12 2_ST25, created May 16, 2020, about 9 KB, which is incorporated by reference herein. In the Sequence Listing:

SEQ ID NO: 1 is the DNA coding sequence for the wildtype KRAS protein.

SEQ ID NO: 2 is the DNA coding sequence for the KRAS G12D protein.

SEQ ID NO: 3 is the DNA coding sequence for the KRAS G12C protein.

SEQ ID NO: 4 is the DNA coding sequence for the KRAS G12V protein.

SEQ ID NO: 5 is the DNA coding sequence for the KRAS G12R protein.

SEQ ID NO: 6 is the DNA coding sequence for the KRAS G12S protein.

SEQ ID NO: 7 is the DNA coding sequence for the KRAS G12A protein.

SEQ ID NO: 8 is the sense strand of the siRNA designed to target wildtype KRAS.

SEQ ID NO: 9 is the antisense strand of the siRNA designed to target wildtype KRAS.

SEQ ID NO: 10 is the sense strand of the siRNA designed to target KRAS G12D.

SEQ ID NO: 11 is the antisense strand of the siRNA designed to target KRAS G12D.

SEQ ID NO: 12 is the sense strand of the siRNA designed to target KRAS G12C.

SEQ ID NO: 13 is the antisense strand of the siRNA designed to target KRAS G12C.

SEQ ID NO: 14 is the amino acid sequence of the HIV-1 Tat cell penetrating peptide.

SEQ ID NO: 15 is the amino acid sequence of the MPG cell penetrating peptide.

SEQ ID NO: 16 is the amino acid sequence of the Pep-1 cell penetrating peptide.

SEQ ID NOs: 17 and 18 are forward and reverse PCR primers for the KRAS transcript.

SEQ ID NOs: 19 and 20 are forward and reverse PCR primers for the Beta-Actin.

### DETAILED DESCRIPTION

### I. Terms

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “comprises” means “includes.” The abbreviation, “e.g.” is derived from the Latin exempli gratia, and is used herein to indicate a non-limiting example. Thus, the abbreviation “e.g.” is synonymous with the term “for example.” In case of conflict, the present specification, including explanations of terms, will control. In addition, all the materials, methods, and examples are illustrative and not intended to be limiting.

Administration: The introduction of a composition into a subject by a chosen route. Administration of an active compound or composition can be by any route known to one of skill in the art. Administration can be local or systemic. Examples of local administration include, but are not limited to, topical administration, intratumoral administration, subcutaneous administration, intramuscular administration, intrathecal administration, intra-ocular administration, topical ophthalmic administration, or administration to the nasal mucosa or lungs by inhalational administration. In addition, local administration includes routes of administration typically used for systemic administration, for example by directing intravascular administration to the arterial supply for a particular organ. Thus, in particular embodiments, local administration includes intra-arterial administration and intravenous administration when such administration is targeted to the vasculature supplying a particular organ. Local administration also includes the incorporation of active compounds and agents into implantable devices or constructs (such as the drug delivery devices described herein), which release the active agents and compounds over extended time intervals for sustained treatment effects. An implantable device is “implanted” by any means known to the art of insertion into the tissue or tissue environment that is the area of a given treatment.

Systemic administration includes any route of administration designed to distribute an active compound or composition widely throughout the body via the circulatory system. Thus, systemic administration includes, but is not limited to intra-arterial and intravenous administration. Systemic administration also includes, but is not limited to, topical administration, subcutaneous administration, intramuscular administration, or administration by inhalation, when such administration is directed at absorption and distribution throughout the body by the circulatory system.

Altered expression: Expression of a biological molecule (for example, RNA (mRNA, miRNA, and the like) or protein) in a subject or biological sample from a subject that deviates from the expression if the same biological molecule in a subject or biological sample from a subject has normal or unaltered characteristics for the biological condition associated with the molecule. Normal expression can be found in a control, a standard for a population, and other similar baseline measures of expression. Altered expression of a biological molecule may be associated with a disease such as a cancer. The term associated with includes an increased risk of developing the disease as well as the disease itself. Expression may be altered in such a manner as to be increased or decreased. The directed alteration in expression of an RNA or protein may be associated with therapeutic benefits resulting from the direct effect on a molecule associated with a pathological condition, or from the indirect effect on such a molecule (e.g. wherein the altered expression results in changes in downstream expression that effect a pathology-related molecule).

Antisense inhibitor: Refers to an oligomeric compound that is at least partially complementary to the region of a target nucleic acid molecule to which it hybridizes. As used herein, an antisense inhibitor or antisense oligonucleotide (also referred to as an “antisense compound”) that is “specific for” a target nucleic acid molecule is one which specifically hybridizes with and modulates expression of the target nucleic acid molecule. As used herein, a “target” nucleic acid is a nucleic acid molecule to which an antisense compound is designed to specifically hybridize and modulate expression. Nonlimiting examples of antisense oligonucleotides include primers, probes, antisense morpholinos, RNA interference (RNAi) agents, such as small (or short) interfering RNAs (siRNAs), micro RNAs (miRNAs), small (or short) hairpin RNAs (shRNAs), and ribozymes. As such, these compounds can be introduced as single-stranded, double-stranded, circular, branched or hairpin compounds and can contain structural elements such as internal or terminal bulges or loops. Double-stranded antisense compounds can be two strands hybridized to form double-stranded compounds or a single strand with sufficient self-complementarity to allow for hybridization and formation of a fully or partially double-stranded compound.

Cancer: The product of neoplasia is a neoplasm (a tumor or cancer), which is an abnormal growth of tissue that results from excessive cell division. Neoplasia is one example of a proliferative disorder. A “cancer cell” is a cell that is neoplastic, for example a cell or cell line isolated from a tumor.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers (such as small cell lung carcinoma and non-small cell lung carcinoma), ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma, astrocytoma, medulloblastoma, craniopharyogioma, ependymoma, pinealoma, hemangioblastoma, acoustic neuroma, oligodendroglioma, menangioma, neuroblastoma and retinoblastoma). In particular embodiments, the cancer that is targeted for treatment by the described compositions and methods is a metastasis, including a solid tumor metastasis, which is not the primary (originating) tumor. It should be noted that although the term metastasis is often used generally for all cancer that has spread from the primary tumor, distinct forms of cancer migration exist which are driven by and respond to distinct biological signals. One example of such distinct migration is regional perineural invasion, which is distinguishable from distant metastases that are typically raised far from the primary tumor as a result of leakage of tumor cells into the blood stream, and are spread by way of the circulatory system.

Examples of hematological tumors include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Chemotherapeutic agent: An anti-cancer agent with therapeutic usefulness in the treatment of diseases characterized by abnormal cell growth or hyperplasia. Such diseases include cancer, autoimmune disease as well as diseases characterized by hyperplastic growth such as psoriasis. One of skill in the art can readily identify a chemotherapeutic agent (for instance, see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993). Non-limiting examples of chemotherapeutic agents include ICL-inducing agents, such as melphalan (Alkeran™), cyclophosphamide (Cytoxan™), cisplatin (Platinol™) and busulfan (Busilvex™, Myleran™). Chemotherapeutic agents include small molecules, nucleic acid, peptide, and antibody-based therapeutic agents; examples of all of which are known in the art. Immunomodulatory agents, which enhance the activity of a subject's immune system against a foreign body, such as a tumor, including a solid tumor, are other examples of chemotherapeutic agents.

Drug Delivery Device (DDD): Device by which a therapeutic agent, such as an antisense inhibitor or chemotherapeutic agent, is provided to a subject. Non-limiting examples of DDDs include drug-eluting implants and stents. The LODER implant is described herein, in particular examples, for use with an RNAi agent, and is an illustrative DDD.

Effective amount of a compound: A quantity of compound sufficient to achieve a desired effect in a subject being treated. An effective amount of a compound can be administered in a single dose, or in several doses, for example daily, during a course of treatment. However, the effective amount of the compound will be dependent on the compound applied, the subject being treated, the severity, and type of the affliction, and the manner of administration of the compound.

Injectable composition: A pharmaceutically acceptable fluid composition comprising at least one active ingredient, for example, a nucleic acid, including an RNAi agent, a peptide, or an antibody. The active ingredient is usually dissolved or suspended in a physiologically acceptable carrier, and the composition can additionally comprise minor amounts of one or more non-toxic auxiliary substances, such as emulsifying agents, preservatives, pH buffering agents and the like. Such injectable compositions that are useful for use with the compositions of this disclosure are conventional; appropriate formulations are well known in the art.

Local Drug EluteR (LODER): Millimeter scale drug delivery insertable device (DDD) or implant, composed of a polymer into which a given drug is incorporated. The drug, such as, but not limited to, an RNAi agent, small molecule, peptide, or antibody, is released into the surrounding environment over a period of time that will vary depending on the LODER composition. For example, in particular embodiments, LODER can release a drug over a period of hours, days, weeks, and even months. In addition to the polymer and a drug, LODER can contain agents which alter (modify) the hydrophobicity and/or pH associated with LODER manufacturing and/or internal environment in-vivo. Examples of particular LODER formulations are described further herein.

MicroRNA (miRNA): Short, single-stranded RNA molecule of typically 18-24 nucleotides long. Endogenously produced in cells from longer precursor molecules of transcribed non-coding DNA, miRNAs can inhibit translation, or can direct cleavage of target mRNAs through complementary or near-complementary hybridization to a target nucleic acid (Boyd, Lab Invest., 88:569-578, 2008). As used herein, a “microRNA sequence” includes both mature miRNA sequences and precursor sequences. As used herein, a microRNA “seed sequence” is a short sequence, generally about seven nucleotides long, that is fully complementary with the target nucleic acid.

Neoplasia, malignancy, cancer and tumor: A neoplasm is an abnormal growth of tissue or cells that results from excessive cell division. Neoplastic growth can produce a tumor. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” Malignant tumors are also referred to as “cancer.”

Perineural invasion (PNI): Also referred to herein as “Regional Perineural Invasion.” Migration of a cancer cell in relation to a nerve and into the space surrounding and/or within the layers of a nerve (the epineurium, perineurium, and endoneurium). The close association with nerve tissue is among the factors that distinguishes PNI from more general definitions of metastasis and cancer cell migration, which occur at a greater distance in the body from a primary tumor. PNI (i.e. regional PNI) is most frequently associated with solid tumors of pancreatic cancer, such as pancreatic ductal adenocarcinoma, gastric carcinoma, colorectal cancer, biliary tract tumors, prostate cancer, cervical cancer, and head and neck cancer. Its occurrence in a patient, often at an early stage of a cancer, is often a sign of poor prognosis, and is often associated with cancer-induced pain.

Pharmaceutical agent: A chemical compound or composition capable of inducing a desired therapeutic or prophylactic effect when properly administered to a subject or a cell. Incubating includes exposing a target to an agent for a sufficient period of time for the agent to interact with a cell. Contacting includes incubating an agent in solid or in liquid form with a cell, such as contacting a tumor with the described siRNA in suspension or as incorporated into a drug delivery device.

Preventing or treating a disease: Preventing a disease refers to inhibiting the development of a disease, for example inhibiting the development of myocardial infarction in a person who has coronary artery disease or inhibiting the progression or metastasis of a tumor in a subject with a neoplasm. Treatment refers to a therapeutic intervention that ameliorates a sign or symptom of a disease or pathological condition after it has begun to develop. In particular examples, treatment of a cancer can include inhibition of progression and/or prevention of a reoccurrence of the disease. In another example, treatment can include sensitizing or predisposing a tumor to an additional treatment, such as an immunomodulatory therapy.

RNA interference (RNA silencing; RNAi): A gene-silencing mechanism whereby specific molecules, such as a double-stranded RNA (dsRNA), trigger the degradation of homologous mRNA (also called target RNA). Double-stranded RNA can be or is processed into small (or short) interfering RNAs (siRNA), which serve as a guide for cleavage of the homologous mRNA in the RNA-induced silencing complex (RISC). The remnants of the target RNA may then also act as siRNA; thus resulting in a cascade effect. An RNAi agent includes any nucleic acid that can either serve directly as siRNA, be processed into siRNA, or produce siRNA, for example DNA that is transcribed to produce RNA that in turn is processed into siRNA.

Sense/anti-sense strand: The strand of dsDNA containing the RNA transcript sequence (read from 5′ to 3′ direction) is the sense strand, and is also known as the “forward” strand. The opposite, reverse-complementary strand, which is used as the template for cellular RNA polymerase, is the antisense strand, and is also known as the “reverse” strand. Likewise, in a dsRNA molecule, the “sense” strand corresponds to the target gene coding sequence, and with the antisense strand, its reverse complement.

Small interfering RNAs: Synthetic or naturally-produced small double stranded RNAs (dsRNAs) that can induce gene-specific inhibition of expression in invertebrate and vertebrate species. These RNAs are suitable for interference or inhibition of expression of a target gene and comprise double stranded RNAs of about 15 to about 40 nucleotides containing a 3′ and/or 5′ overhang on each strand having a length of 0- to about 5-nucleotides, wherein the sequence of the double stranded RNAs is essentially identical to a portion of a coding region of the target gene for which interference or inhibition of expression is desired. The double stranded RNAs can be formed from complementary ssRNAs or from a single stranded RNA that forms a hairpin or from expression from a DNA vector.

Subject: Living multi-cellular organisms, including vertebrate organisms, a category that includes both human and non-human mammals.

Subject susceptible to a disease or condition: A subject capable of, prone to, or predisposed to developing a disease or condition. It is understood that a subject already having or showing symptoms of a disease or condition is considered “susceptible” since they have already developed it.

Target sequence: A target sequence is a portion of ssDNA, dsDNA, or RNA that, upon hybridization to a therapeutically effective oligonucleotide, results in the inhibition of expression of the target.

Therapeutically effective amount: A quantity of compound sufficient to achieve a desired effect in a subject being treated. An effective amount of a compound may be administered in a single dose, or in several doses, for example daily, during a course of treatment. However, the effective amount will be dependent on the compound applied, the subject being treated, the severity and type of the affliction, and the manner of administration of the compound.

Tumor bed: The tissue surrounding a solid tumor.

### II. Overview of Several Embodiments

Described herein is a composition that includes an antisense oligonucleotide agent that targets at least one KRAS mutant allele selected from KRAS G12D, KRAS G12C, KRAS G12V, KRAS G12R, KRAS G12S, and KRAS G12A, for use in inhibiting regional perineural invasion or pain associated with PNI by a solid tumor in a subject.

In a particular embodiment, the solid tumor is of a cancer selected from pancreatic cancer, lung cancer, and colorectal cancer.

In some embodiments, the antisense oligonucleotide agent is an RNA interference (RNAi) agent, which in certain embodiments is a double stranded RNAi agent.

In further particular embodiments, the antisense oligonucleotide agent is an RNAi agent that is siRNA selected from an siRNA having a sense strand set forth herein as SEQ ID NO: 10, and an antisense strand set forth herein as SEQ ID NO: 11, or an siRNA having a sense strand set forth herein as SEQ ID NO: 8, and an antisense strand set forth herein as SEQ ID NO: 9, or siRNA having a sense strand set forth herein as SEQ ID NO: 12, and an antisense strand set forth herein as SEQ ID NO: 13.

In some embodiments, the composition is provided to the subject in a biopolymeric drug delivery device (DDD), such as the DDD described herein as a local drug eluter (LODER), in one or more of any LODER embodiment described herein.

Also described herein is a method for reducing regional perineural invasion by a solid tumor by administering to a subject in need thereof (i.e. a subject diagnosed with a solid tumor or suspected of having a solid tumor or predisposed to having a solid tumor) a therapeutically effective amount of a composition that includes an antisense oligonucleotide agent that targets at least one KRAS mutant allele selected from the group consisting of KRAS G12D, KRAS G12C, KRAS G12V, KRAS G12R, KRAS G12S, and KRAS G12A.

In a particular embodiment of the described method, the solid tumor is of a cancer selected from pancreatic cancer, lung cancer, and colorectal cancer.

In certain embodiments of the described method the antisense oligonucleotide agent is an RNA interference (RNAi) agent, such as a double stranded RNAi agent.

In further particular embodiments of the described method, the antisense oligonucleotide agent is an RNAi agent that is siRNA selected from an siRNA having a sense strand set forth herein as SEQ ID NO: 10, and an antisense strand set forth herein as SEQ ID NO: 11, or an siRNA having a sense strand set forth herein as SEQ ID NO: 8, and an antisense strand set forth herein as SEQ ID NO: 9, or siRNA having a sense strand set forth herein as SEQ ID NO: 12, and an antisense strand set forth herein as SEQ ID NO: 13.

In particular embodiments of the described method the composition is administered to the subject in a biopolymeric drug delivery device (DDD), such as the DDD described herein as a local drug eluter (LODER), in one or more of any LODER embodiment described herein.

In additional embodiments, the methods described herein can also be used for reducing pain associated with regional perineural invasion of a solid tumor, by administering to a subject in need thereof a therapeutically effective amount of a composition comprising an antisense oligonucleotide agent that targets at least one KRAS mutant allele selected from the group consisting of KRAS G12D, KRAS G12C, KRAS G12V, KRAS G12R, KRAS G12S, and KRAS G12A.

Lastly, the compositions described herein can be used in the preparation of a medicament for inhibiting regional perineural invasion or pain associated with a solid tumor in a subject.

### III. Compositions and Methods for Reducing Perineural Invasion

Disclosed herein is the observation that RNAi agents designed to specifically target KRAS G12D or KRAS G12C can, in an illustrative cancer cell line (the pancreatic cancer cell line MIA PaCa-2 which harbors a KRAS G12C mutation), significantly inhibit perineural invasion (PNI). Also disclosed is the observation that an siRNA that is designed to specifically target and knock down expression of the mutant KRAS allele G12D can also significantly knock down expression of the KRAS mutant alleles KRAS G12C, KRAS G12V, KRAS G12R, KRAS G12S, and KRAS G12A.

Accordingly, described herein are compositions and methods for inhibiting and/or preventing regional PNI by use of an antisense oligonucleotide agent, such as an RNAi agent, that inhibits expression of one or more of the noted KRAS G12X alleles: KRAS G12D, KRAS G12C, KRAS G12V, KRAS G12R, KRAS G12S, and KRAS G12A. Such agents can be understood to “target” the noted alleles, even if they are not designed to specifically target a particular mutant allele sequence. For example, as noted, an siRNA designed to specifically target KRAS G12D can also significantly knock down, and therefore target expression of several G12X mutant alleles.

In particular embodiments, the antisense oligonucleotide agent targets a KRAS sequence harboring the mutant allele for KRAS G12D (SEQ ID NO: 2); KRAS G12C (SEQ ID NO: 3); KRAS G12V (SEQ ID NO: 4); KRAS G12R (SEQ ID NO: 5); KRAS G12S (SEQ ID NO: 6); and/or KRAS G12A (SEQ ID NO: 7).

In a further particular embodiment, the antisense oligonucleotide agent is designed to target the KRAS wildtype (WT) sequence (SEQ ID NO: 1).

In particular embodiments, the antisense oligonucleotide agent is an siRNA agent designed to target the KRAS WT sequence, wherein the sense sequence is set forth herein as SEQ ID NO: 8, and the antisense sequence is set forth herein as SEQ ID NO: 9. In other particular embodiments, the antisense oligonucleotide agent is an siRNA agent designed to target the KRAS G12D sequence, wherein the sense sequence is set forth herein as SEQ ID NO: 10, and the antisense sequence is set forth herein as SEQ ID NO: 11. In still other embodiments, the antisense oligonucleotide agent is an siRNA agent designed to target the KRAS G12C sequence, wherein the sense sequence is set forth herein as SEQ ID NO: 12, and the antisense sequence is set forth herein as SEQ ID NO: 13.

The compositions and methods described herein are used to inhibit and/or prevent regional perineural invasion (PNI) and/or pain in a subject through use of an antisense oligonucleotide agent, such as an RNAi agent. In particular embodiments, an RNAi agent for use in the described methods and compositions is a short (or small) interfering RNA (siRNA), short hairpin RNA (shRNA), or microRNA. In other embodiments RNAi agents for use in the described compositions and methods include longer polynucleotide molecules that are processed intracellularly to yield siRNA. Particular examples include DsiRNA, which are cleaved by the RNase III class endoribonuclease dicer into 21-23 base duplexes having a 2-base 3′-overhang; UsiRNAs, which are duplex siRNAs that are modified with non-nucleotide acyclic monomers, termed unlocked nucleobase analogs (UNA), in which the bond between two adjacent carbon atoms of ribose is removed, and which may be designed to enter the RNAi pathway via Dicer enzyme or directly into RISC; self-delivering RNA (sdRNA) such as rxRNA® of RXi Therapeutics, and agents inhibiting the pre-mRNA maturation step of polyA tail addition such as the U1 adaptor (Integrated DNA Technologies (IDT) Inc).

In certain embodiments, the RNAi agent is between 25-30 nucleotides (nt) in length, such as 25-27 nt and 19-25-nt. In other embodiments, the RNAi agent is 19 nt long. In other embodiments, the sense strand and/or the antisense strand further comprises a 1-6-nt 3′-overhang. In particular embodiments, the RNAi agent is 100% complementary to its target sequence. In other embodiments, the RNAi agent is only partially complementary, with 1, 2, 3 or more nucleotides that are different from its target sequence. In other embodiments, a two-base 3′ overhang is present. In more specific embodiments, the sense strand and the antisense strand each further comprises a 2-nt 3′-overhang. In still further embodiments, the 3′ overhangs are made from consecutive deoxythymine (dT) nucleotides, such that a 2 nucleotide 3′ overhang is dTdT (for example in the siRNA sequences set forth herein as SEQ ID NOs 8-13). In other embodiments, a siRNA used in the described methods and compositions has a 19+2 overhang design, namely sense and anti-sense of 19 base-paired nucleotides and two unpaired nucleotides at the 3′ end of each of the strands. In certain embodiments, as exemplified herein, the overhangs are each dTdT.

In other embodiments, one or more nucleotides of the described RNAi agent are modified by 2′-OMe or 2′-F. In particular embodiments, such modifications are made in one or both strands of a described siRNA. The described modified sequences may be used with or, in other embodiments without, overhangs at the 3′ end of each of the strands (in the instance of a dsRNA RNAi agent). In certain embodiments, the overhangs each consist of two unpaired nucleotides. In more specific embodiments, as exemplified herein, the overhangs are each dTdT (2 deoxythymidine residues).

In other embodiments, the described RNAi agents can be chemically modified, separate from or in addition to the modifications described above. In a particular embodiment, the modification is a backbone or linkage modification. In another embodiment, the modification is a nucleoside base modification. In a further embodiment, the modification is a sugar modification. In more specific embodiments, the modification, including the nucleotide modifications described above, is selected from the modifications appearing in Table 1 below. In other embodiments, the modification is selected from a locked nucleic acid (LNA) and/or peptide nucleic acid (PNA) backbone. Other modifications are described in US Patent Application Pub. No. 2011/0195123.

TABLE 1
RNAi agent modifications

| | |
|---|---|
| Modification | Position of the substitution |

Sugar modifications

| | |
|---|---|
| dNTPs- dTdT | 3′-overhangs of sense and/or anti-sense strands |
| dNTPs- dNPs | Any number of residues in the sense strand; 0-4 residues at the |
| | 5′ end of the antisense strand |
| 2′-O-methyl (2′OMe) | Any number of residues in the sense and/or antisense strands |
| rNPs | |
| 2′-fluoro (2′-F) rNPs | Any number of pyrimidine residues in the sense and/or |
| | antisense strands |
| combined use of 2′OMe | Any number of pyrimidine residues in the sense and/or |
| and 2′-F | antisense strands to 2′-F; and any number of purine residues in |
| | the sense and antisense strands to 2′-OMe. |
| 2′-O-(2-methoxyethyl) | Any number of pyrimidine residues in the sense and/or |
| (MOE) rNPs | antisense strands |
| 2′-fluoro-β-D (FANA) | Any number of pyrimidine residues in the sense strand |
| rNPs | |
| Locked nucleic acids | from none till 4 last ribonucleotides at the 3′ end of the sense |
| (LNA) | strand; and 3′ overhangs of the antisense strand |
| combined use of DNA | substitution of any number of pyrimidine (T and C) |
| and 2′-F | ribonucleotides to 2′-F ribonucleotides and any number of |
| | purines (A and G) to deoxyribonucleotides in sense and/or |
| | antisense strands |

phosphate linkage modifications - phosphorothioate (PS)

| | |
|---|---|
| phosphodiester | substitution of any number of ribonucleotides in sense and/or |
| | antisense strands |
| phosphorothioate (PS) | substitution of any number of ribonucleotides in sense and/or |
| | antisense strands |
| boranophosphate DNA | substitution of any number of ribonucleotides in sense and/or |
| or RNA | antisense strands |
| amide-linked | substitution of any number of ribonucleotides in sense and/or |
| | antisense strands |
| phosphoramidate | substitution of any number of ribonucleotides in sense and/or |
| | antisense strands |
| methylphosphonate | substitution of any number of ribonucleotides in sense and/or |
| | antisense strands |
| 2′,5′-linked DNA or RNA | substitution of any number of ribonucleotides in sense strand |

Base_modifications

| | |
|---|---|
| 5-bromouracil (5-Br-Ura) | substitution of any number of ribouracils in sense and/or |
| | antisense strands |
| 5-iodouracil (5-I-Ura) | substitution of any number of ribouracils in sense and/or |
| | antisense strands |
| dihydrouracil | substitution of any number of ribouracils in sense and/or |
| | antisense strands |
| 2-thiouracil | substitution of any number of ribouracils in sense and/or |
| | antisense strands |
| 4-thiouracil | substitution of any number of ribouracils in sense and/or |
| | antisense strands |
| pseudouracil | substitution of any number of ribouracils in sense and/or |
| | antisense strands |
| diaminopurine | substitution of any number of adenines in both sense and/or |
| | antisense strands |
| difluorotoluene | substitution of any number of adenines in both sense and/or |
| | antisense strands |
| peptide nucleic acids | substitution of any number of ribonucleotides in sense and/or |
| (PNAs) | antisense strands |
| (2-aminoethylglycine) | |

modifications to the overhangs and termini

| | |
|---|---|
| 2-nt-3′-DNA overhang | 3′ end of sense and/or antisense strands |
| 2-nt-3′-RNA overhang | 3′ end of sense and/or antisense strands |
| blunt-ended duplexes | 3′ end of sense and/or antisense strands |

chemical conjugation

| | |
|---|---|
| cholesterol | covalently attached to sense and/or antisense strands |
| vitamin-E (α-tocopherol) | covalently attached to sense and/or antisense strands |

In other embodiments, the described RNAi agent may be conjugated to cholesterol, a cell penetrating peptide, or alpha-tocopherol-vitamin E. In certain embodiments wherein the RNAi agent is double-stranded, the cholesterol may be conjugated to the 3′ end of the sense strand. In other embodiments, the cholesterol may be conjugated to the 5′ end of the sense strand. In certain embodiments, in the case of a hairpin-shaped molecule, the cholesterol may be conjugated to the loop. These and further examples of conjugating molecules are described in US Patent Application Pub. No. 2011/0195123.

In certain embodiments, the RNAi agent is associated, either via covalent attachment or via non-covalent complexation, with a cell-penetrating peptide (CPP), also referred to as protein transduction domains (PTDs), which can facilitate the delivery of a molecular cargo to the cytoplasm of a cell. Non-limiting examples of CPP's include HIV-1 Tat (NCBI Gene ID: 155871) or a fragment thereof comprising the sequence YGRKKRRQRRR (SEQ ID NO: 14); pAntp (penetratin) (NCBI Gene ID: 40835); Is1-1 (NCBI Gene ID: 3670); Transportan, Pooga et al), MPG (GALFLGFLGAAGSTMGA; SEQ ID NO: 15); and Pep-1 (KETWWETWWTEW; SEQ ID NO: 16). The sequences and uses of these and other CPPs are known to those skilled in the art.

In other embodiments, the described RNAi agents may be complexed with a cationic molecule, such as DOTAP (N-[1-(2,3-Dioleoyloxy)propyl]-N,N,N-trimethylammonium), DOPE (1,2-dioleoyl-sn-glycero-3-phosphatidylethanolamine), 1,2-dioleoyl-sn-glycero-3-phosphocholine (DOPC), 1,2-di-O-octadecenyl-3-trimethylammonium propane (DOTMA), spermine, PEI (polyethylenimine), a PEI-PLA polymer, or N-Acetylgalactosamine (GalNAc).

In particular embodiments, the RNAi agents are formulated for systemic delivery, in other embodiments, the RNAi agents are formulated for local delivery to an area of treatment, such as in and around the tumor bed of a solid tumor. General methods and formulations for systemic and local delivery of a pharmaceutical agent are referenced above and are well known in the art.

In a particular embodiment of the described methods, the antisense oligonucleotide agent, such as the described RNAi agent is provided to a subject in biopolymeric composition. One such biopolymeric composition for use in local delivery of the antisense oligonucleotide is a biodegradable drug delivery device (DDD, or LODER) which includes an antisense oligonucleotide agent. Such a DDD is implanted into a solid tumor or the surrounding tumor bed such that its antisense payload is released to the tumor or surrounding area. In other particular embodiments, a DDD that delivers the described antisense oligonucleotide agent may be implanted outside of the tumor or tumor bed, provided that it affects the target organ for example, and provides a regional effect.

The DDD of the described compositions and for use in the described methods is generally composed of a biodegradable polymeric matrix; and at least one oligonucleotide agent, such as an RNAi agent, wherein the RNAi agent is incorporated within the biodegradable polymeric matrix.

The described DDD can be a cylinder, a sphere, or any other shape suitable for an implant (i.e. that can be implanted in a subject). In particular embodiments, the DDD is of “millimeter-scale.” That is, a device whose smallest diameter is a least 0.3 mm. In certain embodiments, each of the dimensions (diameter, in the case of a sphere or cylinder; and height and/or width or length, in the case of a cylinder, box-like structure, cube, or other shape with flat walls) is between 0.3-10 mm, inclusive. In other embodiments, each dimension is between 0.5-8 mm, inclusive. In still other embodiments, each dimension is between 0.8-5.2 mm, inclusive, between 1-4 mm, inclusive, between 1-3.5 mm, inclusive, between 1-3 mm, inclusive, or between 1-2.5 mm, inclusive.

In particular embodiments, the device is a cylinder, having a diameter of 0.8 mm. In other preferred embodiments, the cylinder has a length of 5.5 mm. In other embodiments, the cylinder has a diameter of about 0.8 mm and a length of 5.5 mm. In other embodiments, a DDD of the described methods and compositions has the diameter of an 18-gauge needle.

In other embodiments, the volume of the device is between 0.1 mm³ and 1000 mm³, between 0.2 mm³ and 500 mm³, between 0.5 mm³ and 300 mm³, between 0.8 mm³ and 250 mm³, between 1 mm³ and 200 mm³, between 2 mm³ and 150 mm³, between 3 mm³ and 100 mm³, or between 5 mm³ and 50 mm³.

In a particular embodiment, the DDD has a diameter of 0.8 mm and a length of 5.5 mm, containing 25% w/w siRNA, namely about 650 μg of siRNA.

In other embodiments, the w/w agent:polymer load ratio is above 1:100. In more preferred embodiments, the load is above 1:20. In more preferred embodiments, the load is above 1:9. In still more preferred embodiments, the load is above 1:3.

The DDD is composed of polymers, wherein the oligonucleotide agent, such as a siRNA, release mechanism includes both bulk erosion of the polymer and diffusion of the oligonucleotide agent; or in some embodiments, non-degradable, or slowly degraded polymers are used, wherein the main release mechanism is diffusion and the DDD includes surface erosion and/or bulk erosion, and in some embodiments the outer part of the DDD functions as membrane, and its internal part functions as a drug reservoir, which practically is separated and not affected by the surroundings for an extended period (for example from about a week to about a few months). Combinations of different polymers with or without several excipients, with different release mechanisms may also optionally be used. The concentration gradient at the surface is preferably constant during a significant period of the total drug releasing period, and therefore the diffusion rate is effectively constant (termed “zero mode” diffusion). The term “constant” refers to a diffusion rate that is maintained above the lower threshold of therapeutic effectiveness, but which may still optionally feature an initial burst and/or fluctuate, for example increasing and decreasing to a certain degree. In other embodiments, there is an initial burst of less than 10% of the total amount of drug, which may be considered negligible. In other embodiments, there is an initial burst of about 20% of the total amount of drug. In other embodiments, the design enables an initial strong burst of 30% or more of the total amount of drug. The diffusion rate is preferably so maintained for a prolonged period, and it can be considered constant to a certain level to optimize the therapeutically effective period, for example the effective silencing period.

In particular embodiments, the DDD releases the described oligonucleotide agent, such as an RNAi, agent in a controlled fashion, which will vary depending on factors including but not limited to the DDD's constituent polymers, additives, and surface-to-volume ratio. For example, decreasing the surface-to-volume ratio will increase the duration of RNAi agent release time.

The DDDs described herein are designed with a particular drug-release profile. One relevant parameter is the time point at which 95% of the active agent (e.g. the antisense oligonucleotide agent) has been released. In some embodiments, the DDD releases 95% of the active agent in vivo, for example in a human prostate or in a pancreatic tumor, over a time period between 3-24 months inclusive, for example 3, 4, 5, 6, 8, 10, 12, 14, 16, 18, 20, 22, or 24 months and any duration in between, for example 3-12, 2-24, 2-15, or 3-10 months inclusive. Another relevant parameter is the time point at which 90% of the active agent has been released; this may be any of the aforementioned time frames.

Another relevant parameter is the percent of oligonucleotide agent released at a given time point. For example, in some embodiments such as those in which the DDD is releasing an RNAi agent, 80-99% inclusive of the RNAi agent is released 3-months after implantation. In other embodiments, 80-99% of the active agent is released 2, 4, 6, 9, 12, or 24-months after implantation. Alternatively or in addition, in some embodiments no more than 30-50% of the RNAi agent is released from the DDD during the first 3 weeks after implantation. In certain embodiments, less than 5% of the RNAi agent is released from the DDD over a time period of 1 month starting from implantation. In other embodiments, less than 10% of the RNAi agent is released from the DDD over a time period of 1 month starting from implantation.

Delayed-release DDDs are utilized with the described oligonucleotide agents. “Delayed-release”, as used herein, refers to DDDs that do not release more than 10% of the agent within the first 2 months (discounting an initial burst of up to 20%, which sometimes occurs). In other embodiments, the DDD does not release more than 10% of its drug load within the first 3 months. In particular embodiments, DDDs containing 1% trehalose exhibit delayed release.

In other embodiments, the DDD is coated (by dipping, spraying, or any other method known to those skilled in the art) with a slowly-degraded polymer that contains no drug. Various embodiments of slowly-degraded polymers are described herein, each of which can be utilized to create a delayed-release DDD. In some embodiments, the coating comprises a linear-chain monosaccharide; a disaccharide; a cyclic monosaccharide, a cyclic disaccharide. In other embodiments, the coating comprising an additive selected from lactose, sucrose, dextran, and hydroxyethyl starch. In yet other embodiments, the coating comprises mannitol. Alternatively, the coating may comprise trehalose. In still other embodiments, the coating does not comprise a sugar.

The DDD contains a biodegradable polymeric matrix into which the oligonucleotide (e.g. RNAi) agent is incorporated. In particular embodiments, the matrix is composed of poly(lactic acid) (PLA). In other embodiments, the biodegradable matrix is composed of poly(glycolic acid) (PGA). In still other embodiments, the biodegradable matrix comprises the co-polymer of PLA and PGA known as poly(lactic-co-glycolic acid) (PLGA).

PLGA matrices of varying ratios of PLA:PGA are well known and are commercially available. Likewise, methods for making such matrices that incorporate RNAi agents are well known in the art. Exemplary methods (including exemplary methods involving LODER) are described in US Patent Application Pub. No. 2011/0195123. In particular embodiments, the PLA:PGA ratio in the PLGA copolymer is between 95:5 and 5:95, and more particularly between 25:75 and 75:25. In other embodiments, the ratio is between 50:50 and 75:25, meaning that the amount of co-polymer in the DDD includes between 50-75% PLA and between 25-50% PGA. In other embodiments, the PLA:PGA ratio is between 25:75 and 50:50, between 35:65 and 75:25, between 45:55 and 75:25, between 55:45 and 75:25, between 65:35 and 75:25, between 75:25 and 35:65, between 75:25 and 45:55, between 75:25 and 55:45, or between 75:25 and 65:25. In other embodiments, the PLA:PGA ratio is between 80:20 and 90:10, inclusive. In other embodiments, the PLA/PGA ratio is larger than 75:25, between 75:25 and 85:15, or between 75:25 and 95:5. Alternatively, the ratio is smaller than 25:75, between 25:75 and 15:85, or between 25:75 and 5:95. In some embodiments, the co-polymer has a PLA:PGA ratio of between 80:20 and 90:10, inclusive, for example 80:20, 82:18, 84:16, 86:14, 88:12, or 90:10. In other embodiments, the co-polymer has a PLA:PGA ratio larger than 75:25, for example 76:24, 78:22, 80:20, 82:18, 84:16, 86:14, 88:12, 90:10, 92:8, 94:6, 96:4, or 98:2. In yet other embodiments, the co-polymer has a PLA:PGA ratio smaller than 25:75, inclusive, for example 24:76, 22:78, 20:80, 18:82, 16:84, or 14:86, 12:88, 10:90, 8:92, 6:94, 4:96, or 2:98.

In other embodiments the biodegradable polymeric matrix is composed of PEG (poly (ethylene glycol)), which can be the majority of the DDD or used in combination with any other polymer described herein.

Other polymers that can be used in the described DDDs include tri-block PLA-PCL-PLA, wherein PCL denotes poly-caprolactone; Poly(D,L-lactide) (DL-PLA), poly(D,L-glycolide); or poly(D,L-lactide-co-glycolide). Design of biodegradable controlled drug-delivery carriers containing PLA, PGA, PEG, and/or PCL to have a specified release profile are described inter alia in Makadia and Siegel, 2011.

In some embodiments, a polymer used in the described DDDs has a molecular weight (MW) of greater than 5 kilodaltons (kDa). In other embodiments, the MW is greater than 50 kDa. In other embodiments, the MW is greater than 7 kDa, 10 kDa, 15 kDa, 20 kDa, 30 kDa, 70 kDa, 100 kDa, 150 kDa, or greater than 200 kDa. In other embodiments, the MW is between 5-100 kDa, between 7-80 kDa, 10-60 kDa, 20-50 kDa, or between 25-50 kDa. In a particular example extended, slow release (approximately 6 months) can be achieved with a DDD containing PLGA co-polymer having a high PLA:PGA ratio, such as 90:10, and a MW (molecular weight) higher than 50KDa. A similar effect can be achieved by use of PLA.

In other embodiments, the biodegradable matrix further comprises one or more additives for a variety of purposes including modulating hydrophilic-hydrophobic interactions; enabling dispersion of the drug, eliminating aggregation; preserving the drug in hot-temperature or cold-temperature storage conditions; and facilitating creation of cavities in the implant that affect drug diffusion from the matrix.

Hydrophilic-hydrophobic interactions may cause aggregation of the active substance in cases of hydrophilic active substances, such as siRNA, incorporated within a hydrophobic polymer, resulting in aggregation during production or subsequently when the device is implanted into the body of a subject and is subjected for example to hydrolysis. Non-limiting examples of such an additive to reduce such interactions are open monosaccharides, for example mannitol; disaccharides such as trehalose; sorbitol; and other cyclic monosaccharides such as glucose, fructose, galactose and disaccharides such as sucrose or any other cryoprotectant. These additives also in some embodiments function by forming hydrogen bonds with biological molecules as water molecules are displaced, enabling the biological material to retain its native physiological structure and function. The above additives, when chiral, can be in the form of the D-enantiomer, the L-enantiomer, or a racemic mixture. Additional, non-limiting examples of such additives are lactose, sucrose, dextran, and hydroxyethyl starch.

In particular embodiments, the DDD has between 1% and 15% mannitol, such as 1%, 1.5%, 2%, 2.5%, 5%, 7.5%, 10%, or 12.5%, and 15%, or any amount between.

In other particular embodiments, the DDD has less than 5% trehalose, for example in different embodiments 0.5%, 1%, 1.5%, 2%, 2.5%, 3%, 3.5%, 4%, or 4.5%, the effects of which on RNAi agent release can be readily tested.

In other embodiments, the biodegradable matrix comprises an additive for protecting an agent, such as an RNAi agent, against low pH after implantation. The microenvironment in the DDD implant interior tends to be acidic. When delivering an RNAi agent, pH should preferably be maintained above a threshold. For example, polymers including PLGA and oligonucleotides including RNAi drugs might degrade at pH <3. Accordingly, in more specific embodiments such as when the DDD is providing an RNAi agent to the solid tumor or tumor bed, such a pH modulating (i.e. pH-changing) additive may be selected from bicarbonates and carbonates, for example sodium bicarbonate, sodium carbonate, and magnesium hydroxide. In particular examples, sodium bicarbonate is included at a concentration between 0.05% to about 5%, such as about 1%. In other examples, sodium bicarbonate (or other pH modulating agent) is included at less than 1%, including 0.9%, 0.8%, 0.7%, 0.6%, 0.4%, and 0.2% or even less. In still other examples, sodium bicarbonate (or other pH modulating agent) is included at 2%, 3%, 4%, 5%, or any increment in between 1% and 5%.

The described DDDs can contain at least 10 μg of an RNAi agent, such as a siRNA. In other embodiments, the amount is between 10-2000 μg siRNA per device. including between 300-1700 μg siRNA per device, between 300-1100 μg siRNA per device, or between 400-900 μg siRNA per device. In particular embodiments, in addition or as an alternative to the RNAi agent described herein, other therapeutic agents can be incorporated into and delivered by the described DDDs. Non-limiting examples of such agents include an additional RNAi agent targeting other cancer-associated genes; small molecule chemotherapeutic agents, and other biologic immunotherapeutic agents such as but not limited to immunomodulating cytokines and monoclonal antibodies.

It is appreciated that multiple DDDs can be implanted in a given treatment. The amount of the RNAi agent in all the DDD's administered as a batch (a single dose) can be at least 4 μg, for example at least 5 μg, at least 6 μg, at least 7 μg, at least 8 μg, at least 10 μg, at least 12 μg, or at least 15 μg. In still other embodiments, the amount of RNAi agent present per dose is between 2-10 μg, inclusive, for example 2, 3, 4, 5, 6, 7, 8, 9, or 10 μg.

In yet other embodiments, all the DDD's administered as a batch deliver a dose of 0.008-0.065 mg/kg/month, inclusive, for example 0.008 mg/kg/month, 0.01 mg/kg/month, 0.015 mg/kg/month, 0.02 mg/kg/month, 0.03 mg/kg/month, 0.05 mg/kg/month, or 0.065 mg/kg/month.

In certain embodiments, the drug percentage of the described DDDs is at least 20%. In another embodiment, the drug percentage is at least 30%, for example 30%, 35%, 40%, 45%, 50%, 55%, or 60%. In another embodiment, the drug percentage is between 8-30%, inclusive, for example 8%, 9%, 10%, 11%, 12%, 13%, 14%, 15%, 16%, 17%, 18%, 19%, 20%, 22%, 24%, 26%, 28%, or 30%.

As described, a large variety of DDDs can be contemplated, taking the various amounts of polymer, RNAi agent and optional additives. Particular non-limiting examples of such DDDs follow.

In a particular embodiment, the DDD (LODER) contains 64-76% PLGA (with a ratio of PLA:PGA at 90:10); 16-27% RNAi agent; and 5-12% mannitol, with or without .05%-1.5% sodium bicarbonate. In other particular embodiments the DDD can be 80-85% PLGA (with a ratio of PLA:PGA at 85:15); 10-12% siRNA; 7.5-10% mannitol, and 0.1-0.3% sodium bicarbonate.

In still other embodiments, the described DDD contains trehalose and not mannitol. In still other embodiments, the DDD comprises both trehalose and mannitol. In more specific embodiments, the DDD may contain 70-91.2% PLGA; 8-30% siRNA; 0.6-1.5% trehalose; and 0.1-0.4% sodium bicarbonate. In other embodiments, the DDD may contain 75-91.2% PLGA; 8-25% siRNA; 0.6-1.5% trehalose; and 0.1-0.4% sodium bicarbonate. In still other embodiments, the DDD may contain 80-91.2% PLGA; 8-20% siRNA; 0.6-1.5% trehalose; and 0.1-0.4% sodium bicarbonate. In yet other embodiments, the DDD may contain 85-91.2% PLGA; 8-15% siRNA; 0.6-1.5% trehalose; and 0.1-0.4% sodium bicarbonate. In additional embodiments, the DDD may contain 88-91.2% PLGA; 8-12% siRNA; 0.6-1.5% trehalose; and 0.1-0.4% sodium bicarbonate. In yet other embodiments, the DDD may contain 89-91% PLGA; 8-10% siRNA; 0.6-1.5% trehalose; and 0.1-0.4% sodium bicarbonate. In still other embodiments, the DDD may contain about 90% PLGA 85:15, about 9% siG12D, about 1% Trehalose, and about 0.2% NaHCO₃. In any of the above DDD formulations, the siRNA can be replaced by an alternative oligonucleotide agent, such as an alternative nucleic acid.

In other embodiments, the described DDDs can be coated. A coating can be designed for a number of characteristics, including modulating the release rate or preventing protein stickiness during long-term storage. The coating in some embodiments comprises the same material used to form the matrix, for example a PLGA co-polymer matrix with or without the additives or with additives with different ratio but without a oligonucleotide agent (e.g. RNAi agent). In other embodiments, the coating comprises a material similar to that used to form the matrix (for example containing the same building blocks in a different ratio, or containing the same polymer but with a different MW), only without the RNAi agent. In other embodiments, the coating comprises the same material used to form the matrix, together with at least one other polymeric material such as PEG. In other embodiments, the coating includes PLA. In still other embodiments, the coating includes a PLGA co-polymer wherein the PLA:PGA are in a ratio of at least 80:20, for example 80:20, 82:18, 84:16, 85:15, 86:14, 88:12, 90:10, 92:8, 94:6, 96:4, 98:2, and 99:1, and having a MW greater than 50KDa, for example 60KDa, 70KDa, 80KDa, 100KDa, 120KDa, 1500KDa, or 200KDa).

In particular embodiments, the described DDDs also contain oligonucleotide agent-complexed small particles, which are distributed within the biodegradable polymeric matrix of the DDD. Small particles include “microparticles” and “nanoparticles,” Microparticles include particles having a size within the range 800 nm-5 μm (also referred to as microspheres). Nanoparticles include particles of size within the range 4 nm-800 nm. (The lower size of ˜4 nm typifies the smaller particles described here, which in typical embodiments is not a sphere, but a molecular complex, for example a drug molecule such as a siRNA molecule that is complexed with a polymer or conjugated to an additional molecule(s)).

In certain embodiments, the particles comprise a polymeric material as described herein, which can be different from or identical to that in the matrix.

“Different from” refers to a polymer made from different building blocks from that in the matrix or even sharing at least one building block with the polymer in the matrix, but having a different composition. For example, the particles can be composed of PLA, whereas the surrounding matrix of the DDD can be composed of PLGA. In another example the differences between the polymers in the particles and the DDD matrix include polymers containing a particular enantiomer as opposed to a racemic mixture of a given building block (L-PLA vs. DL-PLA), polymers containing the same building blocks in a different ratio (having either the same or different molecular weight (MW)), or containing the same building blocks but having a different MW (having either the same or different ratio). “Identical to” refers to polymers with the same building blocks, in the same ratio, and with the same MW.

It will be appreciated that particles composed of polymers that are “identical to” the constituent polymer of the DDD matrix can contain additional materials that are different from the matrix. In particular embodiments, the polymer in the particles is non-identical to the polymer in the matrix.

In still other embodiments, the small particles do not comprise a polymeric-matrix. For example, the particles may be liposomes. Other examples include particles comprising DOTAP or PEI, or another cationic molecule complexed with the RNAi agent, as similarly described above.

In particular embodiments of DDDs that include agent-complexed small particles, particle complexes, for example siRNA-DOTAP complexes, are dissolved in chloroform and incorporated within larger PLA particles. Such particles are then suspended in ethyl acetate and mixed with PLGA to form a matrix.

In particular examples both the DDD matrix and the small particles are complexed with a RNAi agent. In other examples, the DDD matrix is not complexed with the RNAi agent, but the suspended particles are complexed with the RNAi agent. In those embodiments wherein both the DDD matrix and the particles are complexed with the RNAi agent, the RNAi agent can be the same in the matrix and particles or different in the matrix and particles.

Additional examples of DDDs containing small particles, including constituent components, methods of production and the like, can be found in US Patent Publication No. 2013/0122096, the contents of which are incorporated by reference herein in their entirety.

The methods and compositions described herein are used for inhibiting and/or preventing regional PNI and associated pain by a cancer (e.g. a solid tumor). In particular embodiments, the cancer is a prostate carcinoma. In other nonlimiting embodiments, the cancer is another cancer such as a cancer selected from a pancreatic tumor, a colon tumor, a lung tumor, brain cancer, liver cancer, kidney cancer, melanoma, endometrial carcinoma, gastric carcinoma, renal carcinoma, biliary carcinoma, cervical carcinoma, head and neck cancer, and bladder carcinoma. In more specific embodiments, the cancer is selected from pancreatic carcinoma, pancreatic ductal adenocarcinoma, small-cell lung carcinoma, and colorectal cancer.

In particular embodiments, a mixture of delayed release and non-delayed release DDDs are implanted into the subject. Provision of a combination of delayed-release and non-delayed-release DDD's in some embodiments enables a longer time course of significant chemotherapeutic agent (e.g. siRNA) release, without the need for repeated therapeutic intervention.

In some embodiments, the described DDD is implanted intratumorally. In other embodiments, the DDD is implanted into the vicinity of the tumor. In more specific embodiments, in the case of a well-defined solid tumor, several devices are spaced within the tumor volume. In yet other embodiments, several devices are implanted along a needle cavity within the tumor. In still other embodiments, the device or devices are implanted such that they are not in a direct contact with the perimeter of the tumor. Alternatively, in the case of a poorly defined solid tumor, the device is inserted into an area believed to contain tumor cells.

The following examples are provided to illustrate certain particular features and/or embodiments. These examples should not be construed to limit the disclosure to the particular features or embodiments described.

### EXAMPLES

### Example 1: siG12D Inhibits Nerve Invasion by Pancreatic Cancer Cells in a Dorsal Root Ganglion Model

One of the characteristics of solid tumor metastasis in general, and pancreatic cancer in particular, is the high incidence of perineural invasion (PNI) contributing to the generation of pain that is experienced by cancer patients, such as pancreatic cancer patients. This example tests the effect of an siRNA targeting the KRAS G12D and G12C mutation (siG12D and siG12C) on the migration of pancreatic cancer cells towards neurons.

### Methods

An ex-vivo co-culture model was used to determine whether transfection of MIA PaCa-2 cells with siRNA targeting either KRASG12C or KRASG12D results in reduced migration towards murine DRG (dorsal root ganglia).

Analysis of KRAS mRNA Levels after Transfection with KRASG12C and KRASG12D siRNA

To establish that transfection of siRNA targeting KRASG12D or KRASG12C both down regulate the expression of KRAS at the transcript levels, MIA PaCa-2 cells were plated in duplicates in 12 well plates, at a confluency of 70%. The next day, cells were transfected with indicated concentrations of KRASG12D- or KRASG12C-specific siRNA (siG12D or siG12C, respectively) using lipofectamine 3000 (Invitrogen) as per manufacturer's instructions. For siG12D transfections, anti-Luciferase siRNA was used as a negative control. For transfections with siG12C, non-targeting siRNA from Origene was used. The siRNA targeting KRASG12D has a sense strand set forth herein as SEQ ID NO: 10 (5′ GUUGGAGCUGAUGGCGUAGdTdT 3′), and an antisense strand set forth herein as SEQ ID NO: 11 (5′-CUACGCCAUCAGCUCCAACdTdT-3′). The siRNA targeting KRASG12C has a sense strand set forth herein as SEQ ID NO: 12 (5-′ GUUGGAGCUUGUGGCGUAGdTdT-3′), and an antisense strand set forth herein as SEQ ID NO: 13 (5′-CUACGCCACAAGCUCCAACdTdT-3′). 24 hours after transfection, cells were collected and RNA was purified using the NucleoSpin RNA Plus kit (MACHEREY-NAGEL) as per manufacturer's instructions. 400 ng of RNA were then converted into cDNA using the qScript cDNA Synthesis Kit (Quantabio) as per manufacturer's instructions. We then performed Real time PCR to evaluate the relative quantity of KRAS transcript in cells transfected with siRNA by the delta delta ct method. The following primers were used for amplification of the KRAS transcript: forward: 5′-GAGGCCTGCTGAAAATGACTG-3′ (SEQ ID NO: 17). Reverse: 5′-TTACTACTTGCTTCCTGTAGG-3′(SEQ ID NO: 18). Beta-Actin was used as a house keeping gene internal control: forward primer: 5′-AAATCTGGCACCACACCTTC-3′ (SEQ ID NO: 19), reverse primer: 5′-GGGGTGTTGAAGGTCTCAAA-3′ (SEQ ID NO: 20).

### Analysis of KRAS Protein Levels

Cell extracts were prepared from cells transfected with siRNA directed to KRASG12C or scrambled control siRNA, and collected 48 and 72 hours after transfection. KRAS protein level was assessed by standard Western blot analysis using an anti KRAS antibody (Cell Signaling Technology Ras(27H5) Rabbit mAB, cat#: S3339). Protein extracts were prepared using RIPA buffer. Protein concentration was quantified by Bradford assay and equal amounts were loaded on 12% SDS-PAGE gel and then transferred to a PVDF membrane. ECL was used to detect KRAS protein. As a loading control we used the housekeeping gene Beta-Actin.

### Neural Invasion Ex Vivo Model for Assessment of Nerve—Cancer Cell Interactions

The ex vivo assay technique is described in detail in Na'ara et al.; In Vitro Modeling of Cancerous Neural Invasion: The Dorsal Root Ganglion Model. J Vis Exp. (110): e52990; 2016. Briefly, Mice (C57BL/6J 2-4 weeks old) were killed using CO₂ euthanasia and their excised DRG were implanted approximately 500 μm adjacent to a colony of MIA PaCa-2 cells, in reduced growth factor basement membrane matrix (Cultrex). The MIA PaCa-2 cells were transfected with the indicated siRNAs 48 hours before being transferred to the co-culture plate. Cultures were grown in RPMI-1640, containing 10% FCS in 37° C. and 5% CO₂ incubation conditions. Cultures were examined under the microscope daily, and assessed for neural invasion. On day 13 post implantation, we assessed the percentage of colonies from each treatment that displayed invasion towards the DRG. In another experiment, degree of PNI was observed qualitatively 20-days post implantation.

### Results

### Selection of Cell Line for the Experiments

First, we examined whether our in-house KPC cell line K989 is able to migrate towards DRGs in the DRG ex vivo system, as we previously observed with the human pancreatic cancer cell line MIA PaCa-2. The KRAS gene in K989 bares the G12D mutation and is a better suited target for the siG12D siRNA in the siG12D-LODER. The KRAS gene in the MIA PaCa-2 cells bares the G12C substitution. We could not detect migration of the K989 cells towards the DRG in our experimental system, and so chose MIA PaCa-2 for the remaining experiments discussed herein. As described below and in Example 2, siG12D siRNA effectively knocks-down expression of KRAS G12C, supporting its use as a specific mutant KRAS targeting agent in MIA PaCa-2 cells.

Decrease in KRAS RNA Transcript Kevels after siRNA Transfection

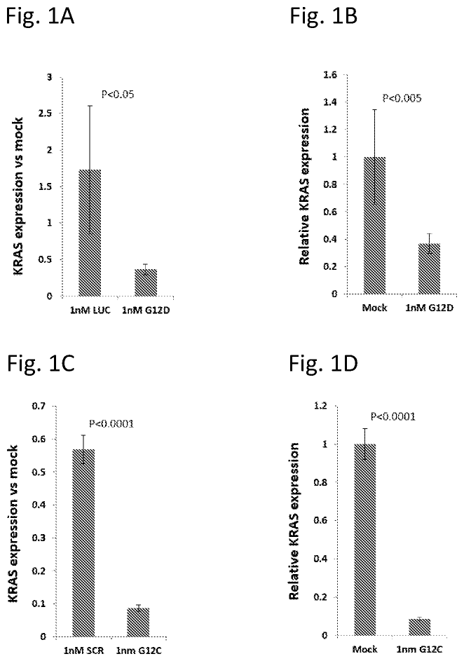

We transfected MIA PaCa-2 cells in triplicate with siRNA directed against the KRASG12D gene (siG12D) (SEQ ID NOs 10 and 11). We tried three different concentrations; 1,100 and 400 nM. We found the maximal decrease in KRAS transcript level was achieved with a concentration of 1 nM siRNA. A modest decrease of about 60% of the mock transfected cells was observed with this concentration. In a separate experiment, we transfected 10 nM of siG12D siRNA and observed a reduction in the transcript levels to -60% of mock transfected cells, similar to the effect observed with 1 nM oligo (FIGS. 1A and 1B).

When we used siRNA designed for the KRASG12C mutation (siG12C) (SEQ ID NOs 12 and 13), we found that the KRAS transcript levels decreased by 91% and 85% compared to mock transfection and scrambled siRNA, respectively (FIGS. 1C and 1D).

Decrease in KRAS Protein Levels after siRNA Transfection

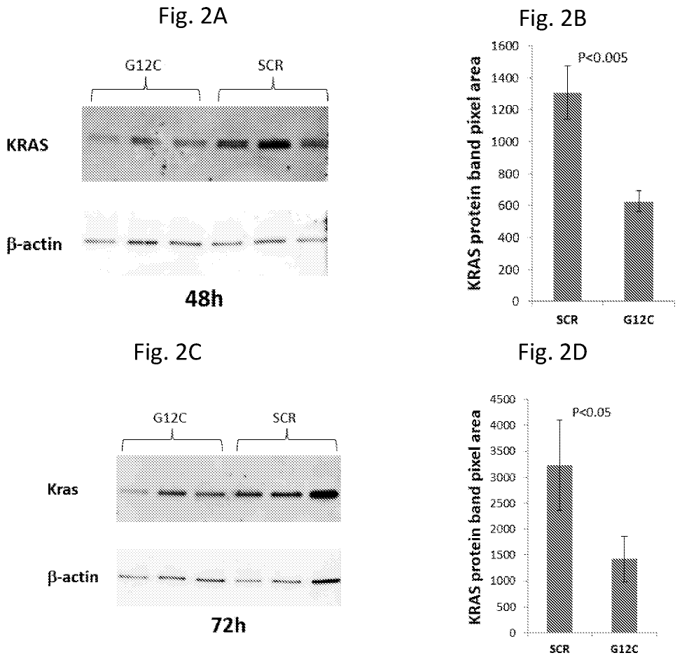

KRAS protein levels in extracts of MIA PaCa-2 cells transfected in triplicates with siRNA directed to KRASG12C (siG12C) or scrambled control siRNA were analyzed by Western Blot. Beta-actin served as a loading control. FIGS. 2A and 2C show the membranes of cell extracts 48 and 72 hours following transfection, respectively, probed with KRAS and Beta-Actin antibodies. FIGS. 2B and 2D show KRAS signal intensity after normalization for Beta-Actin levels, 48 and 72 hours after transfection. The normalized KRAS protein pixel density was approximately half in cell extracts of cells transfected with KRASG12C siRNA compared with cells transfected with scrambled siRNA, 48 hours after transfection (P<0.005) and 72 hours after transfection (P<0.05).

### KRAS Silencing Inhibits Nerve—Cancer Cell Interactions

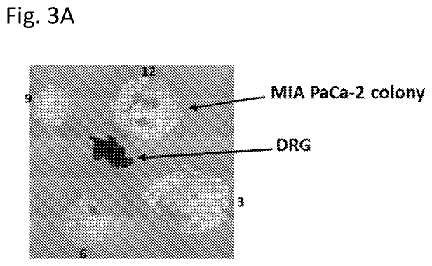

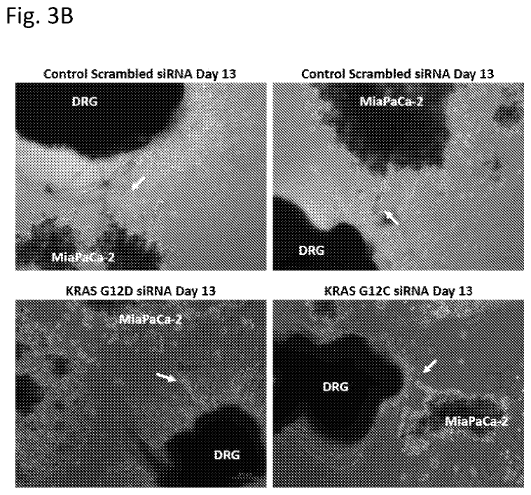

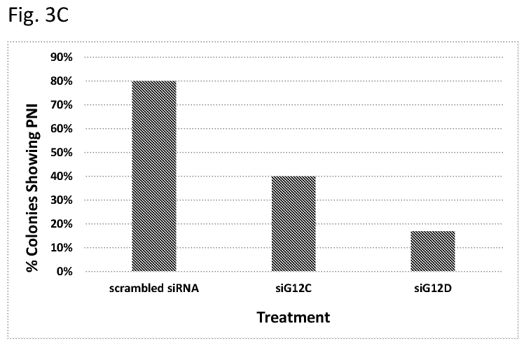

A freshly isolated dorsal root ganglion (DRG) was placed in the middle of a petri dish and four MIA PaCa-2 colonies from the same treatment were plated at 12, 3, 6 and 9 o'clock in relation to the DRG (FIG. 3A). The cells were covered in media and monitored daily for migration of cancer cells from the main colony towards the DRG. Representative images at 13-days post plating are presented in FIG. 3B. The first nerve—cancer cell interaction was observed for the scrambled siRNA-transfected cells on day 7. By day 9 we could also see a neurite-cancer cell interaction in the cells transfected with KRASG12C siRNA. For the KRASG12D siRNA we could observe the first interaction on day 12. Overall, the rate of interactions for the total orientations for each treatment, as measured on day 14, the final day of the experiment, were 80% for the cells treated with scrambled siRNA, 40% for cells treated with KRASG12C siRNA and 17% for cells treated with KRASG12D siRNA. These results are shown in FIG. 3C.

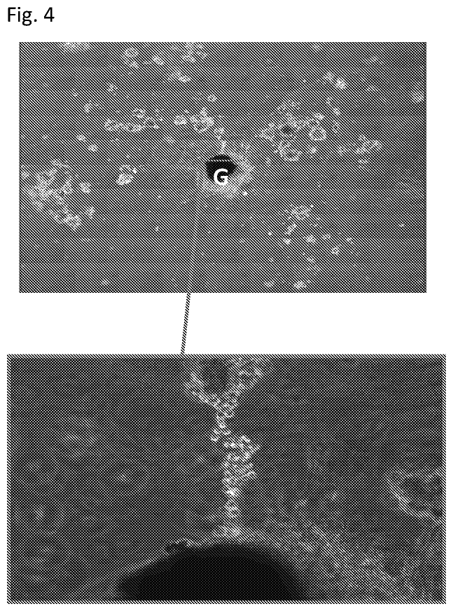

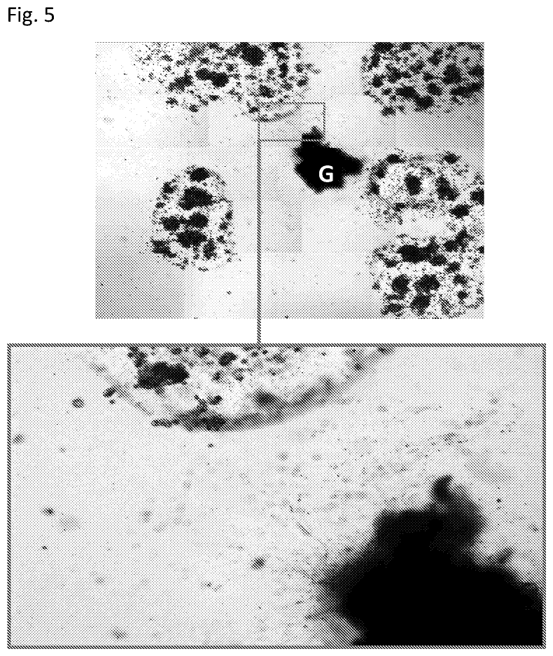

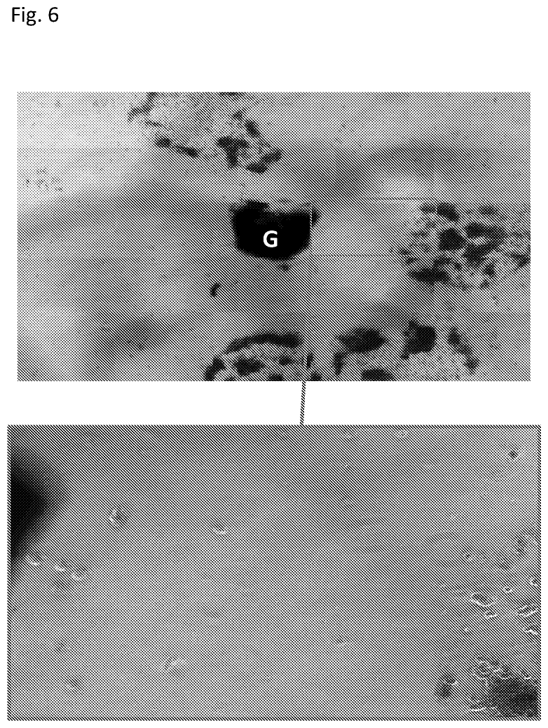

In a separate experiment, transformed MIA PaCa-2 cells were plated as described above, and allowed to co-culture for 20 days. FIGS. 4-6 present representative images taken at the end of this time period. In FIG. 4, PNI from the pancreatic cancer cell colony to the DRG can clearly be seen at both lower (top panel) and higher (bottom panel) magnification. In contrast, as shown in FIG. 5 and FIG. 6, siRNAs targeting KRASG12C and G12D, respectively, significantly inhibit PNI. At lower magnification (top panels in both FIGS. 5 and 6), no migrating cells can be seen connecting the MIA PaCa-2 colonies. At higher magnification (bottom panels), only a few scattered cells are observable apart from the main MIA PaCa-2 colonies.

Cell viability was tested 48 hours after siRNA transfection to verify that inhibition of PNI by siG12C and siG12D was not a result of MIA PaCa-2 cell death (data not shown).

### Discussion

In this example, we were trying to determine whether the silencing of mutant KRAS (KRASG12C in MIA PaCa-2, the cell line tested) can affect cell migration towards DRG in vitro. We used two siRNA oligos; one (siG12D) identical to the formulation of the siG12D-LODER, containing siRNA targeting the KRASG12D mutation that is most abundant in pancreatic ductal adenocarcinoma (PDAC) patients. The other (siG12C) specifically targeting the mutation present in our experimental cell line, MIA PaCa-2, KRASG12C. As expected, we observed better silencing with the KRASG12C oligo. The DRG migration assay is a three dimensional ex-vivo assay often used to evaluate nerve—cancer cell interactions. The results of the experiments suggest that the silencing of mutated KRAS can suppress the neuron-cancer cell interaction and neural invasion as we observed both a delay in the time for interaction and in the rate of colonies that were able to migrate towards the DRG.

### Example 2: siG12D Targets Multiple KRASG12X Mutant Alleles

The previous example showed that an siRNA targeting KRASG12D (siG12D) can also significantly knock down expression of mRNA bearing the KRASG12C mutation. This experiment duplicates and expands this observation, demonstrating the ability for siG12D to knock down expression of multiple mutant alleles. The ability for siRNA targeting wildtype (WT) KRAS to similarly knock down expression of several mutant KRAS alleles is also shown.

### Methods

KRAS allele sequences of interest were based on a KRAS nucleotide sequence (length of 567nt encoding for 128aa from start to stop codon) deposited in the NCBI (National Center for Biotechnology Information) database.

The nucleotide sequences of KRAS WT, G12D, G12C, G12V, G12R, G12S, and G12A are set forth herein as SEQ ID NOs 1-7, respectively. siRNA targeting KRAS G12D is as listed in Example 1. The siRNA targeting KRAS WT has a sense strand set forth herein as SEQ ID NO: 8 (5-′ GUUGGAGCUGGUGGCGUAGdTdT -3′), and an antisense strand set forth herein as SEQ ID NO: 9 (5′-CUACGCCACCAGCUCCAACdTdT-3′).

Hepal-6 cells were cultured using suitable media including 1% Pen/Strep (ATCC CRL-1830, ATCC-formulated Dulbecco's Modified Eagle's Medium supplemented to contain 10% FCS). All cell types used in this study were cultured at 37° C. in an atmosphere with 5% CO2 in a humidified incubator.

Mouse Hepal-6 cells were seeded at a density of 20.000 cells/well in white-walled, 96-well tissue culture plates followed by co-transfection of cells with siRNAs of interest together with one of seven different Dual-Glo reporter plasmids. Constructs are based on psiCHECK-2 vectors from Promega containing KRAS (and derivative) sequences in 3′-UTR context of R-Luc (namely reporter constructs for KRAS WT, G12D, G12V, G12A, G125, G12C, and G12R). Co-transfection of cells with siRNAs and Dual-Glo reporter constructs was carried out using Lipofectamine2000 (Invitrogen/Life Technologies) according to the manufacturer's instructions. Dose-response experiments were done with siRNA concentrations of 10, 2.5, 0.625, 0.156, 0.039, 0.0098, 0.0024, 0.0006, 0.00015 and 0.000038 nM. Appropriate positive and negative controls were used. For each siRNA and control, at least four wells were transfected in parallel, and individual data points were collected from each well.

The Dual-Glo luciferase assay was performed according to the manufacturer's instructions. Luminescence was read using a 1420 Luminescence Counter (WALLAC

VICTOR Light, Perkin Elmer, Rodgau-Jügesheim, Germany) following incubation in the presence of substrate in the dark. For each well with KRAS siRNA treated cells, R-Luc activity was normalized to F-Luc activity, relative to the mean R-Luc/F-Luc activities in mock or negative control siRNA treated cells. In other words, the activity of any siRNA was expressed as percent R-Luc activity (normalized to F-Luc activity) in treated cells, relative to the mean R-Luc activity (normalized to F-Luc activity) across control wells.

### Results

Hepal-6 cells were transfected with three siRNAs of interest (targeting KRAS WT, KRAS G12D, and F-Luc) together with each and every Dual-Glo reporter plasmid in a dose-response setup. Final concentrations of siRNAs were 10 nM, going down in nine 4-fold dilution steps to 3.8E-05 nM. All data were generated in quadruplicates, cells were again incubated for 24 hours post-transfection followed by the Dual-Glo luciferase assay. Summary results of these assays are presented in Table 2. As presented in the table, siG12D can effectively target expression of the assayed KRAS mutant alleles to varying extents from 63.8% (G125) to 95.8% (G12D). While significant, the knock down activity of siG12D against the G12C allele was relatively modest (65% decrease in expression). However, as demonstrated in Example 1, this targeting activity was able to effectively inhibit PNI of the G12C-harboring MIA PaCa-2 cell colonies.

In addition to the knock down efficacy of siG12D against all of the mutant alleles tested, Table 2 also shows that siRNA targeting WT KRAS was able to effectively knock down mutant allele expression.

TABLE 2
Knock-down activity of KRAS siRNAs against mutant alleles

| KRAS | | IC20 | IC50 | IC80 | Max. Inhib. |
|---|---|---|---|---|---|
| Reporter | siRNA | [nM]: | [nM]: | [nM]: | [%]: |
| WT | KRAS | 0.002 | 0.008 | 0.039 | 93.3 |
| | G12D | | | | |
| WT | KRAS | 0.002 | 0.010 | 0.050 | 95.2 |
| | WT | | | | |
| WT | F-Luc | #N/A | #N/A | #N/A | 3.7 |
| G12D | KRAS | 0.001 | 0.006 | 0.031 | 95.8 |
| | G12D | | | | |
| G12D | KRAS | 0.002 | 0.010 | 0.070 | 90.5 |
| | WT | | | | |
| G12D | F-Luc | #N/A | #N/A | #N/A | 5.2 |
| G12V | KRAS | 0.006 | 0.051 | #N/A | 70.6 |
| | G12D | | | | |
| G12V | KRAS | 0.003 | 0.021 | 0.993 | 81.7 |
| | WT | | | | |
| G12V | F-Luc | #N/A | #N/A | #N/A | 5.4 |
| G12A | KRAS | 0.003 | 0.018 | 0.188 | 86.9 |
| | G12D | | | | |
| G12A | KRAS | 0.004 | 0.027 | 2.219 | 81.2 |
| | WT | | | | |
| G12A | F-Luc | #N/A | #N/A | #N/A | 7.8 |
| G12S | KRAS | 0.010 | 0.089 | #N/A | 63.8 |
| | G12D | | | | |
| G12S | KRAS | 0.002 | 0.007 | 0.049 | 89.8 |
| | WT | | | | |
| G12S | F-Luc | #N/A | #N/A | #N/A | 0.5 |
| G12C | KRAS | 0.007 | 0.093 | #N/A | 65.0 |
| | G12D | | | | |
| G12C | KRAS | 0.002 | 0.008 | 0.055 | 90.1 |
| | WT | | | | |
| G12C | F-Luc | #N/A | #N/A | #N/A | 6.3 |
| G12R | KRAS | 0.007 | 0.066 | #N/A | 68.5 |
| | G12D | | | | |
| G12R | KRAS | 0.002 | 0.009 | 0.068 | 88.8 |
| | WT | | | | |
| G12R | F-Luc | #N/A | #N/A | #N/A | 3.7 |

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1.-8. (canceled)

9. A method for reducing regional perineural invasion by a solid tumor comprising:
administering to a subject a therapeutically effective amount of a composition comprising an antisense oligonucleotide agent that targets at least one KRAS mutant allele selected from the group consisting of KRAS G12D, KRAS G12C, KRAS G12V, KRAS G12R, KRAS G12S, and KRAS G12A.

10. The method of claim 9, wherein the solid tumor is of a cancer selected from the group consisting of pancreatic cancer, lung cancer, and colorectal cancer.

11. The method of claim 9, wherein the antisense oligonucleotide agent is an RNA interference (RNAi) agent.

12. The method of claim 11, wherein the RNAi agent is a double stranded RNAi agent.

13. The method of claim 11, wherein the RNAi agent comprises a siRNA having a sense strand set forth herein as SEQ ID NO: 10, and an antisense strand set forth herein as SEQ ID NO: 11.

14. The method of claim 9, wherein the composition is administered to the subject in a biopolymeric drug delivery device.

15. The method of claim 14, wherein the biopolymeric drug delivery device is a local drug eluter (LODER).

16. A method for reducing pain associated with regional perineural invasion of a solid tumor comprising:
administering to a subject a therapeutically effective amount of a composition comprising an antisense oligonucleotide agent that targets at least one KRAS mutant allele selected from the group consisting of KRAS G12D, KRAS G12C, KRAS G12V, KRAS G12R, KRAS G12S, and KRAS G12A.

17. (canceled)

18. The method of claim 16, wherein the composition is administered to the subject in a biopolymeric drug delivery device.

19. The method of claim 18, wherein the biopolymeric drug delivery device is a local drug eluter (LODER).
